(19) United States
(12) Patent Application Publication
(10) Pub. No.: US 2005/0229937 A1
(43) Pub. Date: Oct. 20, 2005

(54) Mayo stand covers and methods and apparatus for forming the same

(76) Inventors: Gary D. Salvaggio, Flat Rock, NC (US); Bart D. Salvaggio, Hendersonville, NC (US)

Correspondence Address: MYERS BIGEL SIBLEY & SAJOVEC, PO BOX 37428, RALEIGH, NC, 27627, US

(21) Appl. No.: 11/107,238

(22) Filed: Apr. 15, 2005

Related U.S. Application Data
(60) Provisional application No. 60/563,053, filed on Apr. 16, 2004.

(51) Int. Cl. A61F 5/37; A61B 19/00

(52) U.S. Cl. 128/849

(57) ABSTRACT

A Mayo stand cover for use with a Mayo stand includes a flat, tubular bag body including first and second opposed layers defining a bag cavity therebetween. The bag body has a closed first end and an open second end. The first and second layers include first and second end edges, respectively, defining an opening at the second end. The opening and the bag cavity are sized and configured to receive the Mayo stand. The Mayo stand cover further includes a cuff assembly. The cuff assembly includes at least one cuff member separately formed from the bag body and secured to each of the first and second layers adjacent the first and second end edges such that the at least one cuff member defines a cuff cavity with each of the first and second opposed layers.

This application claims the benefit of priority from U.S. Provisional Patent Application No. 60/563,053, filed Apr. 16, 2004, the disclosure of which is incorporated herein by reference in its entirety.

### FIELD OF THE INVENTION

### BACKGROUND OF THE INVENTION

Mayo stands are used during surgical procedures to hold surgical instruments, bandages, etc. Typically, a Mayo stand includes a horizontal tray or table surface supported by and cantilevered from a vertical leg that extends to a base. The Mayo stand may include casters.

In order to ensure a sterile surface, the Mayo stand tray is generally covered with a Mayo stand cover, which may be disposable. Conventional Mayo stand covers take the form of a tubular bag closed at one end and open at the opposite end. The open end is slid over the tray and the vertical leg until the portion of the Mayo stand cover adjacent the closed end covers the tray and the open end surrounds the vertical support. A patch or layer of an absorbent material may be provided on the outer surface of the Mayo stand cover and positioned such that it overlies the tray.

In order to allow a user to install the Mayo stand cover without contacting the outer surface or the user's hands with the Mayo stand, which may be non-sterile, it is known to provide Mayo stand covers including a cuff surrounding the open end of the Mayo stand cover. The cuff is formed by rolling the open end of the Mayo stand cover back over the body of the Mayo stand cover. The user may insert his or her hands into the cuff to open the Mayo stand cover and to position the Mayo stand cover on the Mayo stand. Prior to use, the body of the Mayo stand cover can be folded and tucked into the cuff for storage and handling.

### SUMMARY OF THE INVENTION

According to embodiments of the present invention, a Mayo stand cover for use with a Mayo stand includes a flat, tubular bag body including first and second opposed layers defining a bag cavity therebetween. The bag body has a closed first end and an open second end. The first and second layers include first and second end edges, respectively, defining an opening at the second end. The opening and the bag cavity are sized and configured to receive the Mayo stand. The Mayo stand cover further includes a cuff assembly. The cuff assembly includes at least one cuff member separately formed from the bag body and secured to each of the first and second layers adjacent the first and second end edges such that the at least one cuff member defines a cuff cavity with each of the first and second opposed layers.

According to further embodiments of the present invention, a method for forming a Mayo stand cover includes: forming a flat, tubular bag body including first and second opposed layers defining a bag cavity therebetween, the bag body having a closed first end and an open second end, the first and second layers including first and second end edges, respectively, defining an opening at the second end, wherein the opening and the bag cavity are sized and configured to receive the Mayo stand; and securing at least one cuff member to each of the first and second layers adjacent the first and second end edges such that the at least one cuff member defines a cuff cavity with each of the first and second opposed layers, wherein the at least one cuff member is separately formed from the bag body.

According to further embodiments of the present invention, an apparatus for forming a Mayo bag includes a bag body forming station and a cuff assembly forming station. The bag body forming station is operative to form a flat, tubular bag body including first and second opposed layers defining a bag cavity therebetween, the bag body having a closed first end and an open second end, the first and second layers including first and second end edges, respectively, defining an opening at the second end, wherein the opening and the bag cavity are sized and configured to receive the Mayo stand. The cuff assembly forming station is operative to secure at least one cuff member to each of the first and second layers adjacent the first and second end edges such that the at least one cuff member defines a cuff cavity with each of the first and second opposed layers, wherein the at least one cuff member is separately formed from the bag body.

According to further embodiments of the present invention, a Mayo stand cover for use with a Mayo stand includes a flat, tubular bag body including first and second opposed layers defining a bag cavity therebetween and opposed side edges. The bag body has a closed first end and an open second end. The first and second layers include first and second end edges, respectively, defining an opening at the second end. The opening and the bag cavity are sized and configured to receive the Mayo stand. The Mayo stand cover further includes a cuff assembly. The cuff assembly includes at least one cuff member joined to the bag body and defining a cuff cavity with the first layer. The cuff cavity extends outwardly beyond at least one of the side edges of the bag body to define a gap between the at least one cuff member and the at least one of the side edges of the bag body.

According to further embodiments of the present invention, a method for forming a Mayo stand cover includes forming a flat, tubular bag body including first and second opposed layers defining a bag cavity therebetween and opposed side edges, the bag body having a closed first end and an open second end, the first and second layers including first and second end edges, respectively, defining an opening at the second end, wherein the opening and the bag cavity are sized and configured to receive the Mayo stand; and forming at least one cuff member joined to the bag body and defining a cuff cavity with the first layer, wherein the cuff cavity extends outwardly beyond at least one of the side edges of the bag body to define a gap between the at least one cuff member and the at least one of the side edges of the bag body.

According to further embodiments of the present invention, an apparatus for forming a Mayo bag includes a bag body forming station and a cuff assembly forming station. The bag body forming station is operative to form a flat, tubular bag body including first and second opposed layers defining a bag cavity therebetween and opposed side edges, the bag body having a closed first end and an open second end, the first and second layers including first and second end edges, respectively, defining an opening at the second end, wherein the opening and the bag cavity are sized and configured to receive the Mayo stand. The cuff assembly forming station is operative to form at least one cuff member joined to the bag body and defining a cuff cavity with the first layer, wherein the cuff cavity extends outwardly beyond at least one of the side edges of the bag body to define a gap between the at least one cuff member and the at least one of the side edges of the bag body.

Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is a front, top perspective view of a Mayo stand cover according to embodiments of the present invention;

FIG. 2 is an rear, bottom perspective view of the Mayo stand cover of FIG. 1;

FIG. 3 is a top plan view of the Mayo stand cover of FIG. 1;

FIG. 4 is a cross-sectional view of the Mayo stand cover of FIG. 1 taken along the line 4-4 of FIG. 3;

FIG. 5 is a front, top perspective view of the Mayo stand cover of FIG. 1 in an unfolded position;

FIG. 6 is a flow chart representing methods according to method embodiments of the present invention for forming a Mayo stand cover;

FIG. 7 is a schematic diagram of an apparatus according to embodiments of the present invention for forming a Mayo stand cover;

FIG. 8 is a front, top perspective view of a bag body for forming a Mayo stand cover in accordance with method embodiments of the present invention;

FIG. 9 is a top plan view of the bag body of FIG. 8 in a folded position;

FIG. 10 is a schematic diagram of a cuff forming station in accordance with embodiments of the present invention; and

FIG. 11 is a fragmentary view of an intermediate construction in accordance with methods of the present invention.

### DETAILED DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that when an element is referred to as being “coupled” or “connected” to another element, it can be directly coupled or connected to the other element or intervening elements may also be present. In contrast, when an element is referred to as being “directly coupled” or “directly connected” to another element, there are no intervening elements present. Like numbers refer to like elements throughout. As used herein the term “and/or” includes any and all combinations of one or more of the associated listed items.

In addition, spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly.

Well-known functions or constructions may not be described in detail for brevity and/or clarity.

As used herein the expression “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

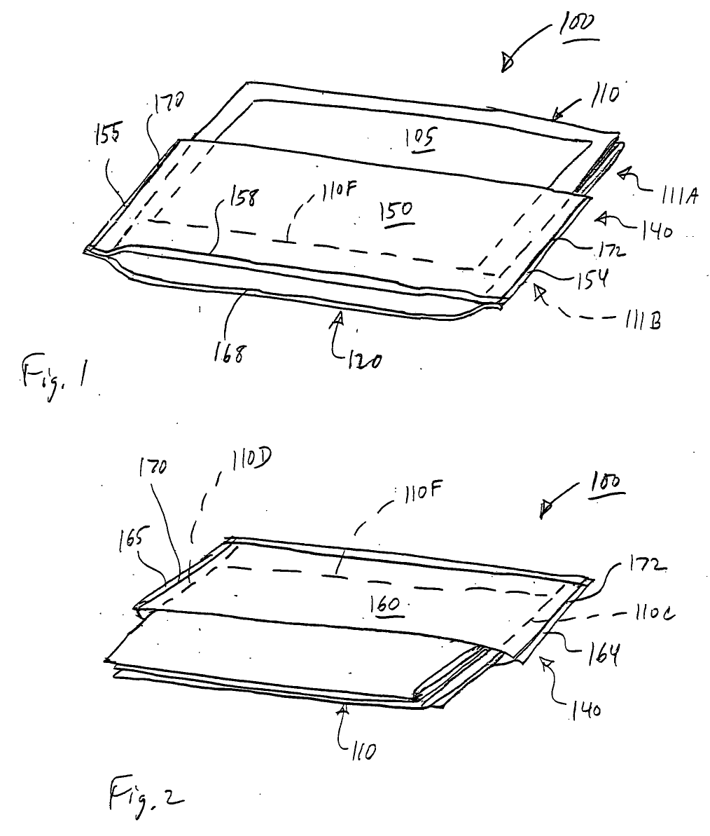

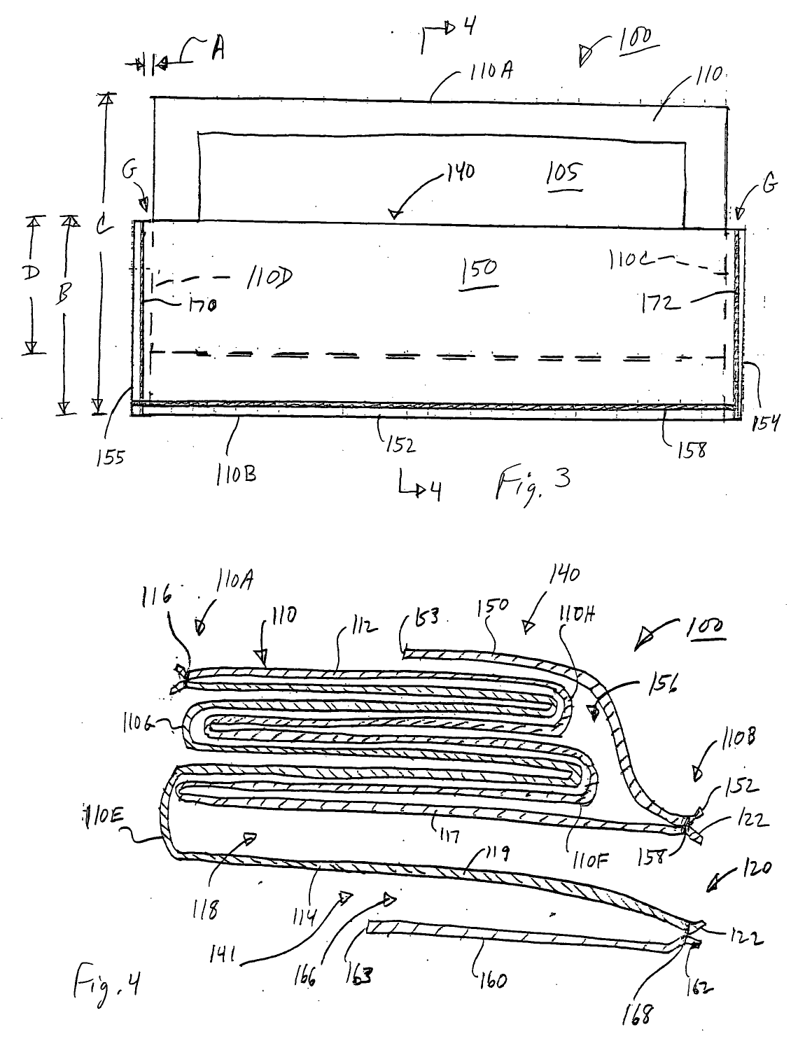

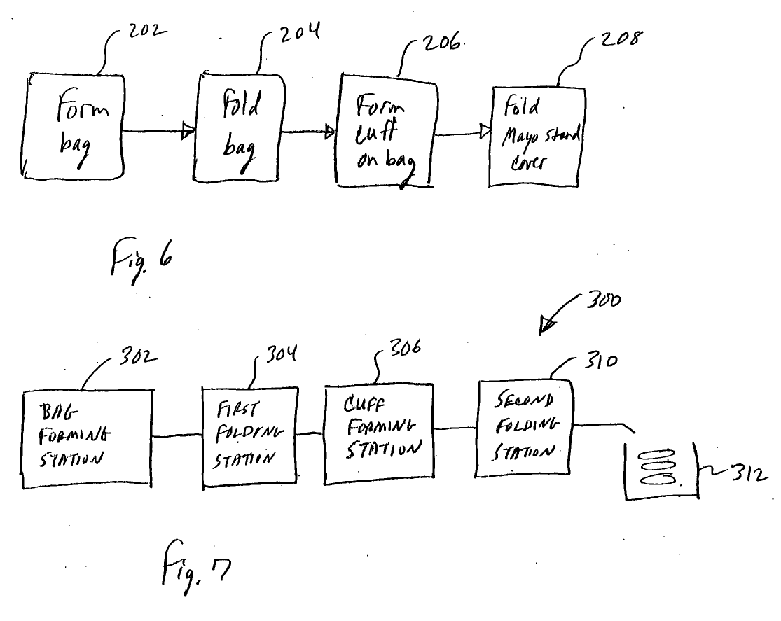

With reference to FIGS. 1-4, a Mayo stand cover 100 according to embodiments of the present invention is shown therein. In FIG. 5, the Mayo stand cover 100 is shown in an unfolded condition (as a Mayo stand cover 100′). The Mayo stand cover 100 may be adapted to be operatively placed over a Mayo stand of conventional or other construction.

The Mayo stand cover 100 includes a bag body 110 and a cuff assembly 140. The cuff assembly 140 includes cuff members 150 and 160. The bag body 100 and the cuff member 150 define a first (typically top) cuff cavity portion 156. The bag body 110 and the cuff member 160 define a second (typically bottom) cuff cavity portion 166. The bag body 110 is folded and positioned such that a portion 111B thereof is disposed in the cavity portion 156 and a portion 111A extends outwardly from the cavity portion 156. In this manner, the cuff assembly 140 holds the bag body 110 in a closed position. A user can slip his or her hands into the cavity portion 156 and/or the cavity portion 166 to handle the Mayo stand cover 100 during installation of the Mayo stand cover on a Mayo stand, for example.

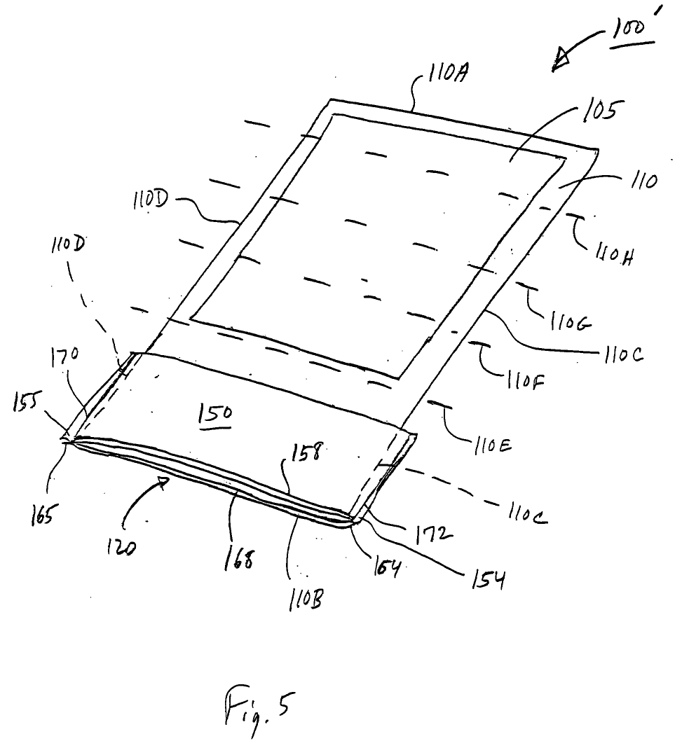

Turning to the bag body 110 in more detail, the bag body 110 includes a tubular bag with opposed, closed side edges 110C, 110D. The side edges 110C, 110D may be closed by seams or seals (e.g., thermo-sealed), or the side edges 110C, 110D may be formed by folds in the bag body 110 (as shown). In particular, the bag body 110 may be formed from an extended tube that is folded flat so as to form the side edges 110C, 110D. The bag body 110 also includes opposed ends 110A, 110B. The end 110A is closed by a seal 116. The end 110B has width-wise extending edges 122 defining an opening 120. The side and end edges define a first layer (e.g., a top layer) 112 and a second layer (e.g., a bottom layer) 114. The layers 112, 114 define a cavity 118 therebetween that is in communication with the opening 120. The bag body 110 is sized and configured to fit over the tray and vertical leg or support of a Mayo stand. As shown, the bag body 110 is folded in an accordion fashion about fold lines 110E, 110F, 110G, 110H. A layer or patch 105 of absorbent material (e.g., a non-woven material) can be bonded to the top panel 112 (not shown in FIG. 4).

The cuff members 150, 160 are separately formed from and secured to the bag body 110. The cuff member 150 extends fully across the width of the layer 112 and the cuff member 160 extends fully across the width of the bottom panel 114. The cuff member 150 and the end most panel 117 define the cavity portion 156. The cuff member 160 and the end most panel 119 define the cavity portion 166.

The cuff member 150 has a lower edge 152, an upper edge 153, and opposed side edges 154, 155. The lower edge 152 is secured to the bag body 110 at or adjacent the edge 122 of the layer 112 by a seal or seam 158. The upper edge 153 is free. Similarly, the cuff member 160 has a lower edge 162, an upper edge 163, and opposed side edges 164, 165. The lower edge 162 is secured to the bag body 110 at or adjacent the edge 122 of the layer 114 by a seal or seam 168. The edge 163 is free.

The side edges 154, 164 and the side edges 155, 165 are outwardly spaced apart from the side edges 110C and 110D, respectively. The cuff members 150 and 160 are secured to one another at or adjacent the edges 154, 164 by a seam or seal 172. The cuff members 150 and 160 are secured to one another at or adjacent the edges 155, 165 by a seam or seal 170. The seals 170, 172 extend generally parallel to and are outwardly spaced apart from the edges 110D and 110C, respectively, to define gaps G (see FIG. 3). According to some embodiments, each of the gaps G has a width A of at least 0.5 inch. According to some embodiments, each gap G has a width A of between about 0.5 and 1.5 inches. According to some embodiments, the gap G is not provided and the seals 170, 172 extend generally immediately adjacent and alongside the respective edges 110D, 110C. The seals 170, 172 do not include the bag body 110 (i.e., the cuff members 150, 160 are not directly secured to the bag body 110 by the seals 170, 172).

From the foregoing, it will be appreciated that the cuff assembly 140 formed by the cuff members 150 and 160 fully surrounds the bag body 110 adjacent the open end of the bag body 110 and defines a circumscribing cavity 141 including both of the cavity portions 156 and 166. Accordingly, the Mayo stand cover 100 can be used in the same manner as conventional Mayo stand covers of the type having a folded back cuff. Moreover, by forming the cuff assembly 140 wider than the bag body 110, clearance is provided between the edge 110C and the seal 172 and between the edge 110D and the seal 170. This clearance may allow for greater ease and effectiveness in handling. The clearance may provide an enlarged effective opening 120 for inserting the tray. This clearance may also reduce or eliminate bunching of the folded portion of the bag body 110 that is inserted into the cuff assembly 140. As a result, the Mayo stand cover 100 may lay more flat and provide an improved appearance.

According to some embodiments, the height B of the cuff assembly 140 is at least 70% of the full height C of the folded Mayo stand cover 100. According to some embodiments, the height B is between about 2 and 10 inches. According to some embodiments, the lower most fold (e.g., the fold 100F) of the bag body 110 extends a depth of at least 2 inches into the cavity portion 156.

The bag body 110 and the cuff assembly 140 can be formed of any suitable material. For example, the bag body 110 and the cuff assembly 140 may be formed of fluid impervious and/or polymeric films such as polyethylene, TYVEK™ or any other suitable material. According to some embodiments, the bag body 110 and the cuff assembly 140 are preferably formed of polyethylene. The bag body 110 and the cuff assembly 140 may be formed of different materials from one another. The cuff members 150 and 160 may be formed of different materials from one another.

The bag body 110 may be folded in a different configuration than that shown herein.

As shown, a fringe or flashing extends outwardly beyond the seals 158, 168, 170, 172. Some or all of this flashing may be eliminated so that the edges of the respective layers are directly mated by seals. Seals can be formed by any suitable means, including hot melt adhesive, tape, and/or ultrasonic welding. According to some embodiments, the seals are thermoformed by thermal impulse sealing. The seals can be rounded or angled, for example, at the front corners of the cuff assembly 140. According to some embodiments, the seals 158, 170, and 172 collectively form a single, continuous seal and, likewise, the seals 168, 170 and 172 form a single, continuous seal.

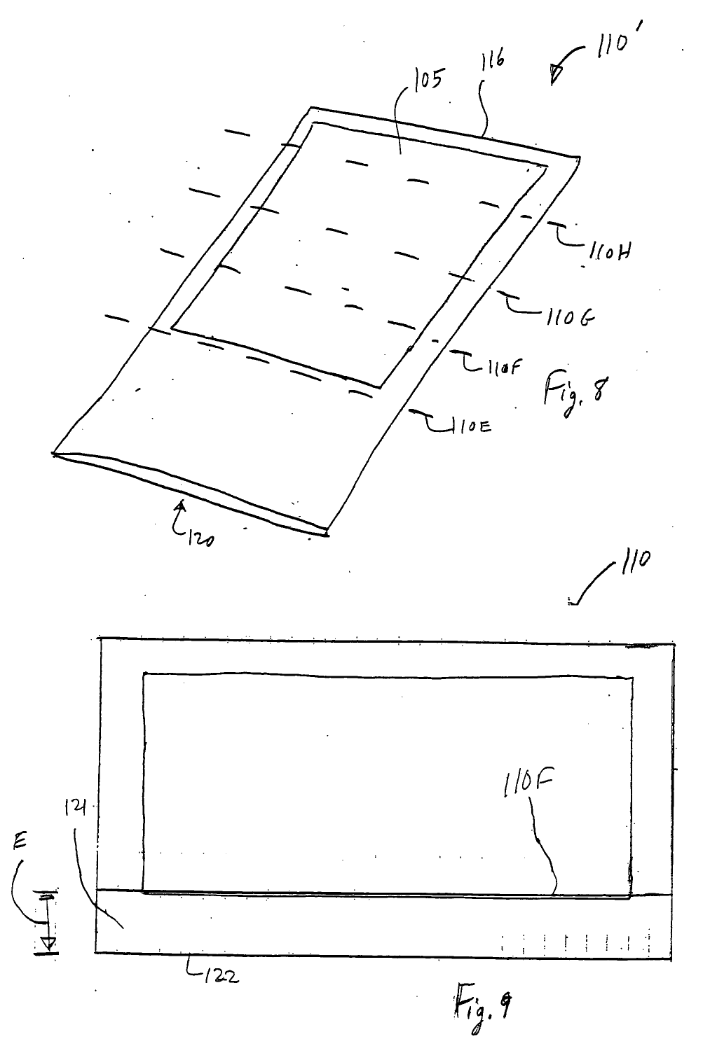

Mayo stand covers according to the present invention may provide benefits and improvements in manufacture as well. In particular, Mayo stand covers according to the present invention may facilitate more efficient, effective and/or complete automated manufacture. With reference to FIGS. 6 and 7, in accordance with embodiments of the present invention and using apparatus 300 according to embodiments of the present invention, a Mayo stand cover such as the Mayo stand cover 100 may be formed as follows. Each of the stations discussed below may comprise one or more stations, and the methods and apparatus 300 may include a continuous process or apparatus or, alternatively, may include multiple separate and discontinuous steps and apparatus. The Mayo stand covers may be formed serially and some operations may be executed in parallel.

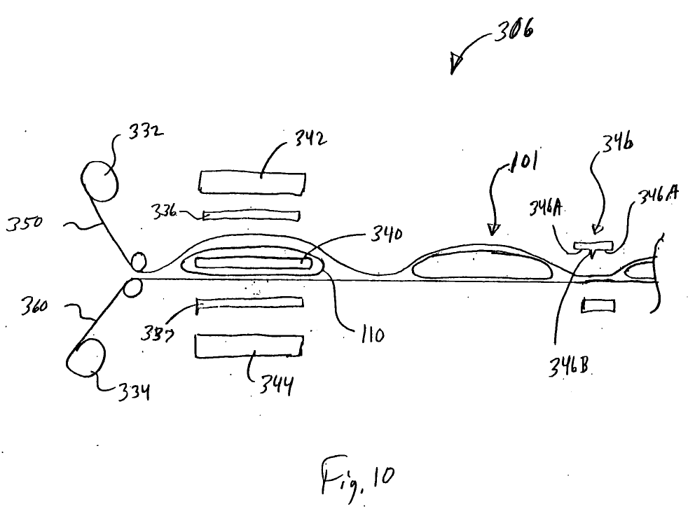

A bag body 110′ as shown in FIG. 8 is formed by a bag forming station 302 (Block 202). As shown, the bag body 110′ so formed includes an end seal 116 and an absorbent supplemental layer or patch 105.

The bag body 110′ is folded by a first folding station 304 about the fold lines 110E, 110F, 110G, 110H to form the bag body 110 as shown in FIG. 9 (Block 204). The folds may be formed parallel to the direction of conveyance, i.e., in the machine direction. The bag body 110 so formed may include a marginal portion or tail 121 adjacent the front end edges 122. The marginal portion 121 includes portions of the top and bottom layers 112, 114 extending below the frontmost fold 110F. According to some embodiments, the marginal portion 121 has a height E of at least 0.5 inch. According to some embodiments, the marginal portion 121 has a height E of between about 0.5 and 3 inches.

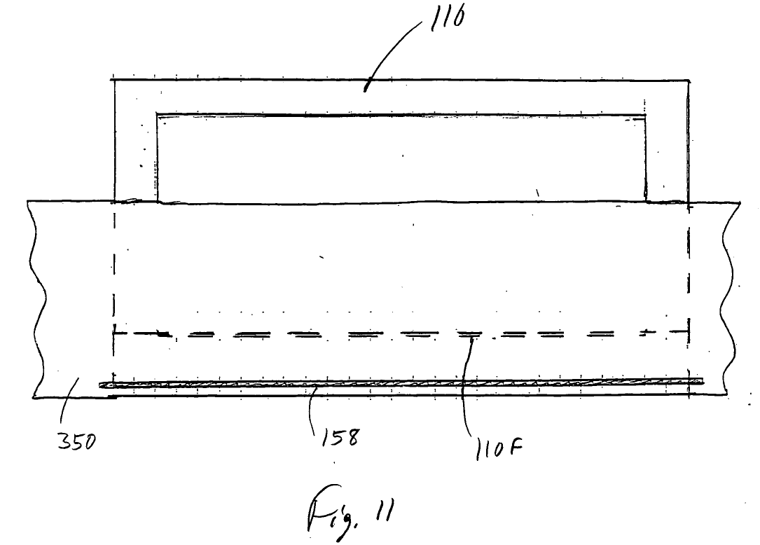

The folded bag body 110 passes through a cuff forming station 306 to form the cuff assembly 140 on the bag body 110 (Block 306). In accordance with certain embodiments, the cuff forming station 306 is configured as shown in FIG. 10. In the cuff forming station 306 of FIG. 10, the bag body 110 is transported on a conveyor (not shown). A continuous web 350 corresponding to the cuff member 150 is provided from a reel 332 and a continuous web 360 corresponding to the cuff member 160 is provided from a reel 334. The open end of the bag body 110 is pulled open by a vacuum manifold 336 (which engages the layer 112) and a vacuum manifold 337 (which engages the layer 114) and an insulator plate or paddle 340 is inserted into the bag body 110 between the layers 112, 114. At least a portion of the insulator paddle 340 extends along the respective end edges 122. The insulator paddle 340 may be formed of aluminum covered with Teflon™. The webs 350 and 360 are registered with the edges 122 of the layers 112 and 114, respectively, and thermal impulse sealers 342 and 344 are converged into contact with the webs 350 and 360, respectively, to form the opposed seals 158 and 168 as shown in FIG. 11. The insulator paddle 340 serves as a bearing surface for each of the thermal impulse sealers 342, 344 while preventing the layers 112, 114 from being sealed to one another. The insulator paddle 340 may be cooled by a fluid circulated through the paddle and/or through a handle or handles supporting the paddle 340.

The intermediate structure 101 including the bag body 110 attached to the webs 350, 360 by the seals 158, 168 is released by the vacuum heads 336, 337 and the bag body 110 and the webs 350, 360 are pulled or conveyed to a side sealing station 346. The side sealing station 346 forms the side edge seal 172 using a thermal impulse sealer 346A and cuts the webs 350, 360 to form the side edges 154, 164 using a blade 346B. The intermediate structure 101 is further conveyed and the thermal impulse sealer 346A forms the side edge seal 170 and the blade 346B cuts the webs 350, 360 to form the side edges 155, 165. According to some embodiments and as shown, the impulse sealer 346A includes upstream and downstream portions that simultaneously form the seal 172 on an upstream intermediate structure 101 and the seal 170 on a downstream intermediate structure 101, respectively. The blade 346B may be omitted and the thermal impulse sealer 346A may be used to form both the seals and to cut the webs 350, 360. Further, multiple side sealing stations 346 may be employed so that the side seals 170 and 172 are simultaneously formed and/or the webs 350, 360 are simultaneously cut on either side of the bag body 110. The Mayo stand cover 100 as shown in FIGS. 1-5 is thereby formed.

The Mayo stand cover 100 may thereafter be transported to a second folding station 310 where the Mayo stand covers are folded traversely to the folds 110E, 11F, 110G, 110H (Block 208). One or more such folds may be made and these folds may be the final folds formed. The Mayo stand covers 100 may thereafter be stacked in a hopper 312 or the like.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention. Therefore, it is to be understood that the foregoing is illustrative of the present invention and is not to be construed as limited to the specific embodiments disclosed, and that modifications to the disclosed embodiments, as well as other embodiments, are intended to be included within the scope of the invention.

1. A Mayo stand cover for use with a Mayo stand, the Mayo stand cover comprising:
a) a flat, tubular bag body including first and second opposed layers defining a bag cavity therebetween, the bag body having a closed first end and an open second end, the first and second layers including first and second end edges, respectively, defining an opening at the second end, wherein the opening and the bag cavity are sized and configured to receive the Mayo stand; and
b) a cuff assembly including at least one cuff member separately formed from the bag body and secured to each of the first and second layers adjacent the first and second end edges such that the at least one cuff member defines a cuff cavity with each of the first and second opposed layers.

2. The Mayo stand cover of claim 1 wherein at least a folded portion of the bag body is in a folded position and at least a portion of the folded portion is disposed in the cuff cavity and retained in the folded position by the at least one cuff member.

3. The Mayo stand cover of claim 1 wherein:
the at least one cuff member includes first and second cuff members;
the first cuff member is secured to the first layer along the first edge; and
the second cuff member is secured to the second layer along the second edge.

4. The Mayo stand cover of claim 3 wherein the first and second cuff members each have side edges and are secured to one another, but not the bag body, along their respective side edges.

5. The Mayo stand cover of claim 1 wherein:
the bag body has opposed side edges; and
the at least one cuff member surrounds the side edges of the bag body so as to to define a gap between the at least one cuff member and each of the side edges of the bag body.

6. The Mayo stand cover of claim 1 wherein the at least one cuff member is secured to each of the first and second layers by a thermoformed seal.

7. A method for forming a Mayo stand cover, the method comprising:
a) forming a flat, tubular bag body including first and second opposed layers defining a bag cavity therebetween, the bag body having a closed first end and an open second end, the first and second layers including first and second end edges, respectively, defining an opening at the second end, wherein the opening and the bag cavity are sized and configured to receive the Mayo stand; and
b) securing at least one cuff member to each of the first and second layers adjacent the first and second end edges such that the at least one cuff member defines a cuff cavity with each of the first and second opposed layers, wherein the at least one cuff member is separately formed from the bag body.

8. The method of claim 7 including:
providing at least one continuous cuff web;
securing at least one portion of the at least one cuff web to the first and second layers adjacent the first and second end edges; and
severing the at least one portion of the at least one cuff web from a remaining portion of the at least one cuff web.

9. The method of claim 7 including:
providing first and second continuous cuff webs;
securing a first portion of the first cuff web to the first layer adjacent the first end edge;
securing a second portion of the second cuff web to the second layer adjacent the second end edge;
severing the first and second portions of the first and second cuff webs from remaining portions of the first and second cuff webs.

10. The method of claim 7 wherein the step of securing the at least one cuff member to each of the first and second layers adjacent the first and second end edges includes:
inserting an insulator member between the first and second layers;
thermoforming a first seal between the at least one cuff member and the first layer;
thermoforming a second seal between the at least one cuff member and the second layer; and
wherein the first and second seals are formed from opposite sides of the bag body and are each formed using the insulator paddle.

11. (canceled)

12. A Mayo stand cover for use with a Mayo stand, the Mayo stand cover comprising:
a) a flat, tubular bag body including first and second opposed layers defining a bag cavity therebetween and opposed side edges, the bag body having a closed first end and an open second end, the first and second layers including first and second end edges, respectively, defining an opening at the second end, wherein the opening and the bag cavity are sized and configured to receive the Mayo stand; and
b) a cuff assembly including at least one cuff member joined to the bag body and defining a cuff cavity with the first layer, wherein the cuff cavity extends outwardly beyond at least one of the side edges of the bag body to define a gap between the at least one cuff member and the at least one of the side edges of the bag body.

13. The Mayo stand cover of claim 12 wherein at least a folded portion of the bag body is in a folded position and at least a portion of the folded portion is disposed in the cuff cavity and retained in the folded position by the at least one cuff member.

14. The Mayo stand cover of claim 12 wherein the at least one cuff member is separately formed from the bag body.

15. (canceled)

16. (canceled)
